SEQUENCE LISTING (4 sequences)

SEQ ID NO: 1 - protein, 37 aa, Homo sapiens
EVVPPQVLSEPNEEAGAALSPLPEVPPWTGEVSPAQR

SEQ ID NO: 2 - protein, 37 aa, Pongo pygmaeus
EVVPPQVLSEQNEEAGAALSPLPEVPPWTGEVSPAQR

SEQ ID NO: 3 - protein, 37 aa, Macaca mulatta
EVVPPQVLREQNEEAGAALSPLPEVPPWTGDVSPAQR

SEQ ID NO: 4 - protein, 37 aa, Felis catus
EVVPPQVLSEQNEEAGAALSPLPEVPPWAGEVNPAQR

(19) United States
(12) Patent Application Publication
(10) Pub. No.: US 2015/0174201 A1
(43) Pub. Date: Jun. 25, 2015

(54) THERAPEUTIC METHOD FOR TREATING CONGESTIVE HEART FAILURE

(71) Applicant: Madeleine Pharmaceuticals Pty Ltd, Mount Barker (AU)

(72) Inventor: Thomas Robert Geimer, Mount Barker (AU)

(73) Assignee: Madeleine Pharmaceuticals Pty Ltd

(21) Appl. No.: 14/575,606

(22) Filed: Dec. 18, 2014

Related U.S. Application Data
(63) Continuation of application No. 14/000,789, filed on Aug. 21, 2013, now Pat. No. 9,018,168.
(60) Provisional application No. 61/445,826, filed on Feb. 23, 2011.
(60) Provisional application No. 61/445,814, filed on Feb. 23, 2011.

(51) Int. Cl. A61K 38/17

(52) U.S. Cl. CPC A61K 38/1709

(57) ABSTRACT

The present invention relates to a method of treating congestive heart failure (CHF) in a subject comprising administering a peptide derived from atrial natriuretic peptide (ANP) prohormone (eg vessel dilator; VSDL) or a mimetic thereof. In a particular application, the invention provides a method of treating the particular indication known as acute decompensated congestive heart failure (ADCHF). Devices for intravenous or subcutaneous infusion for use in the method of the invention are also disclosed.

### INCORPORATION BY REFERENCE

This patent application is a continuation of U.S. patent application Ser. No. 14/000,789, which is the National Stage of International Patent Application No. PCT/AU2011/001026, filed Aug. 11, 2011, which claims priority from U.S. Provisional Patent Application No. 61/445,814 titled “Therapeutic method 2” filed 23 Feb. 2011, and U.S. Provisional Patent Application No. 61/445,826 titled “Therapeutic method 3” filed 23 Feb. 2011. The entire content of these applications is hereby incorporated by reference.

In addition, the following patent is referred to herein: U.S. Pat. No. 5,691,310 titled “Methods of treatment using proANF peptides”. The entire content of this patent is hereby incorporated by reference.

### FIELD OF THE INVENTION

The present invention relates to a method of treating congestive heart failure (CHF) in a subject. In a particular application, the invention provides a method of treating the particular indication known as acute decompensated congestive heart failure (ADCHF).

### SEQUENCE LISTING

The sequences listed in the accompanying Sequence Listing are presented in accordance with 37 C.F.R. 1.822. The Sequence Listing is submitted as an ASCII computer readable text file, which is incorporated by reference herein.

### BACKGROUND TO THE INVENTION

CHF is a major health problem affecting over 23 million people worldwide causing significant morbidity and mortality′. CHF occurs when the heart inadequately pumps oxygenated blood to the rest of the body, resulting in the retention of fluid (oedema), shortness of breath (dyspnea) and lethargy. Often, it is also accompanied by renal failure as the kidneys strain to maintain glomerular filtration in an attempt to excrete the build up of excess fluids². CHF may be caused by one or a combination of factors including: weakened heart muscle, damaged heart valves, blocked blood vessels supplying the heart muscle (ie cardiac arteries), high blood pressure leading to a thickening of the heart muscle (ie left ventricular hypertrophy), congenital heart disease, prolonged arrhythmias, infections, and life style factors such as smoking, obesity and alcohol or drug (eg cocaine) consumption which can, for example, be the cause of long term, uncontrolled high blood pressure (hypertension) that may damage the heart muscle and blood vessels. If steps are not taken to treat CHF when it becomes apparent and/or modify patient life style, the CHF may become a long term (ie chronic) condition. The severity of symptoms of patients with chronic CHF can progress to a level where there is a “marked limitation in activity [and the patient is] comfortable only at rest” as per Class III of the New York Heart Association (NYHA) CHF functional classification. Consequently, patients with chronic CHF that has progressed to NYHA class III can require frequent hospitalisation and/or palliative care. Moreover, while such chronic CHF is generally stable, it may easily decompensate to the often critical state known as acute decompensated congestive heart failure (ADCHF). This can be caused for a variety of reasons including arrhythmias, ischaemia, illness such as pneumonia and poor compliance with diet and medications¹⁹.

In the United States alone, over 1.1 million people are admitted to hospital every year with ADCHF¹, 10% of which die in care and 40% of which die within the year. Further, it has been reported that over 6.5 million days of hospitalisation per year in the United States are attributable to ADCHF³. These costs account for almost 50% of the US$34 billion spent each year on heart failure care in the United States. Unfortunately, ADCHF remains as an unmet clinical need, with current monotherapeutic and polytherapeutic treatments being effectively, and efficaciously, limited at alleviating symptoms only.

Vessel dilator (VSDL) is a naturally occurring 37 amino acid (aa) cardiac peptide consisting of amino acids 31-67 of the 126 aa atrial natriuretic peptide (ANP) prohormone^{4, 6 and 7}. Like other natriuretic peptides, the main biological activity of VSDL is to regulate blood pressure and maintain plasma volume in healthy individuals by mediating natriuretic, diuretic and haemodynamic effects⁴. Preliminary studies on VSDL's effect in CHF have been conducted via both preclinical and human clinical studies^{8,9}. One of the first studies exploring the use of synthetic VSDL in a CHF animal model was in normal and compensated aorto-caval (AV) fistula dogs, resulting in similar significant reductions in arterial blood pressure, right atrial pressure and elevations in urinary sodium excretions. The human studies have involved patients with chronic, but stable, New York Heart Association (NYHA) Class III CHF¹⁰, and have shown that VSDL can significantly improve natriuretic, diuretic and haemodynamic parameters without any symptomatic side effects. In particular, it has been shown that VSDL is able to increase urine flow 2- to 13-fold, and urine flow was still increased (P<0.001) 3 hours after the VSDL infusion was halted¹⁰. Further, it was found that VSDL enhanced sodium excretion 3- to 4-fold in CHF patients (P<0.01), and was still significantly (P<0.01) elevated 3 hours after infusion. Moreover, it was found that VSDL could: decrease systemic vascular resistance (SVR) (24%); decrease pulmonary vascular resistance (PVR) (25%); decrease pulmonary capillary wedge pressure (PCWP) (33%); decrease central venous pressure (CVP) (27%); increase cardiac output (CO) (34%); increase cardiac index (CI) (35%); and increase stroke volume index (SVI) (24%); without significantly affecting heart rate or pulmonary artery pressure in the CHF patients. In addition, it is also known that VSDL promotes the synthesis of the renal protective agent, prostaglandin E₂ (PGE₂), and can increase circulating PGE₂ 8-fold compared to basal levels¹¹. PGE₂ also benefits CHF patients by decreasing mean arterial pressure and total peripheral resistance while increasing stroke volume index and cardiac output.

It has been reported that VSDL circulates in healthy humans at significantly higher basal levels than other atrial natriuretic peptides, including ANP and B-type natriuretic peptide (BNP), where the circulating concentration of VSDL is 17-fold and 48-fold higher than ANP and BNP respectively. Hitherto the studies described herein, this was believed to owe, in part, to VSDL possessing a significantly longer circulatory t_{1/2} of ˜120 minutes and biological t_{1/2} of ˜>6 hours. In comparison, it has been reported that BNP has a circulating t_{1/2} of ˜3.1 minutes and biological t_{1/2} of ˜<½ hour^{4, 6-9, 12 and 13}. In patients with volume overload, including those with CHF, circulating natriuretic peptides are elevated. This is thought to be indicative of a compensatory mechanism attempting to reinstate homeostasis. However, in severe congestion, it appears that the body is unable to produce sufficient quantities to restore balance.

The present applicant has now recognised that the benefits demonstrated in preclinical and clinical use of VDSL in the treatment of CHF are superior to those seen with the current natriuretic treatments for ADCHF¹⁴, and hereby proposes VSDL administration to patients with chronic CHF or ADCHF as a safe and effective treatment for mediating beneficial haemodynamic effects with additional beneficial natriuretic, diuretic and renal effects, whilst regulating plasma volume and blood pressure (BP) within clinically acceptable ranges and without seriously adverse side effects. It is also anticipated that such VSDL treatment may reduce hospitalisation time and readmission rates for chronic CHF and ADCHF patients. Further, the present applicant has found that effective treatment of chronic CHF and ADCHF patients with VSDL can be achieved with doses that are substantially lower than expected.

### SUMMARY OF THE INVENTION

In its broadest aspect, the present invention provides a method of treating chronic congestive heart failure (CHF) or acute decompensated congestive heart failure (ADCHF) in a subject, said method comprising administering to the subject an effective amount of a peptide derived from atrial natriuretic peptide (ANP) prohormone or a mimetic thereof.

Preferably, the method is used for treating chronic CHF with a symptom severity level of NYHA class III or, more preferably, ADCHF with a symptom severity level of NYHA class III or IV (ie where the subject shows “severe limitations [and] experiences symptoms even while at rest”).

The method may be suitable for the treatment of non-human primates and other mammals such as livestock (including race horses), exotic animals (eg tigers and elephants) and companion animals (eg dogs and cats), however typically the subject will be a human, in which case, the method is most preferably used for treating ADCHF characterised by:

1. Dyspnoea at rest or dyspnoea with minimal activity (ie difficulty breathing at rest while sitting, or difficulty breathing while lying flat or with one pillow, or difficulty breathing with minimal activity such as talking, eating); and
2. At least one of the following signs:
Tachypnoea with respiratory rate >20 breaths per minute, or
Pulmonary congestion/oedema with rales or crackles/crepitations at least a third above lung bases; and
   3. At least one of the following objective measures:
Pulmonary oedema/congestions as shown by chest X-ray, or
Circulating B-type natriuretic peptide (BNP) concentration of ≧400 pg/ml or circulating NT-proBNP concentration of ≧1000 pg/ml, or
A PCWP >20 mmHg or
A systolic dysfunction within the last 12 months (EF<50%) as determined by Trans-Thoracic Echocardiogram (TTE), nuclear testing, cardiac magnetic resonance imaging (MRI) or ventricular angiography).

The term “effective amount” as used herein refers to an amount of the peptide or mimetic that will elicit a desired biological or therapeutic response in the subject (eg beneficial haemodynamic effects with, preferably, additional beneficial natriuretic, diuretic and renal effects whilst regulating plasma volume and blood pressure (BP) within clinically acceptable ranges). The effective amount will, however, typically be in the range of 0.1 to 75 μg/kg/day. The effective amount of the peptide or mimetic is administered by infusion (preferably, by intravenous (iv) or subcutaneous (sc) infusion) as a single dose per day or, otherwise, across multiple times per day (eg 2 to 4 times per day). The effective amount of the peptide or mimetic may, for example, be administered by infusion at a rate of <100 ng/kg/min, and preferably, at a rate of about ≦75 ng/kg/min, more preferably at a rate of about ≦50 ng/kg/min or ≦30 ng/kg/min, still more preferably at a rate of about ≦25 ng/kg/min, even still more preferably ≦20 ng/kg/min and, and yet still more preferably, at a rate of about ≦15 ng/kg/min.

In a further aspect, the present invention provides the use of a peptide derived from atrial natriuretic peptide (ANP) prohormone or a mimetic thereof in the manufacture of a medicament for infusion of the peptide or mimetic, for treating chronic congestive heart failure (CHF) or acute decompensated congestive heart failure (ADCHF) in a subject.

In a still further aspect, the present invention provides an infusion device containing, or provided with, a medicament comprising a peptide derived from atrial natriuretic peptide (ANP) prohormone or a mimetic thereof adapted for infusion of the peptide or mimetic, for treating chronic congestive heart failure (CHF) or acute decompensated congestive heart failure (ADCHF) in a subject.

### BRIEF DESCRIPTION OF THE ACCOMPANYING FIGURES

FIG. 1 provides tabulated response data for three ADCHF patients infused intravenously (iv) with 12.5 ng/kg/min for 60 minutes recorded from commencement of infusion (0 minutes) to 5 days post-infusion as indicated;

FIGS. 2A-2E provide graphical representations of the results of FIG. 1: (FIG. 2A) Mean arterial pressure (MAP), (FIG. 2B) Pulmonary capillary wedge pressure (PCWP), (FIG. 2C) Cardiac index (CI), (FIG. 2D) Systemic vascular resistance (SVR), and (FIG. 2E) NT-proBNP circulatory levels (ng/L); and

FIG. 3 provides a graphical representation of measured VSDL concentrations in blood samples taken at the various time points, as corrected for a baseline (ie endogenous) VSDL concentration of 0.024 ng/ml.

### DETAILED DESCRIPTION OF THE INVENTION

The present invention relates to the use of a peptide derived from atrial natriuretic peptide (ANP) prohormone (eg VSDL) or a mimetic thereof, in a method for the treatment of chronic CHF or ADCHF in a subject comprising administering to a subject an effective amount of the peptide or mimetic (typically in the range of 0.1 to 75 μg/kg/day).

In a first embodiment, the present invention provides a method of treating chronic congestive heart failure (CHF) or acute decompensated congestive heart failure (ADCHF) in a subject, said method comprising administering to the subject an effective amount of a peptide derived from atrial natriuretic peptide (ANP) prohormone or a mimetic thereof by infusion at a rate of 1 to 400 ng per kg subject body weight per minute (ng/kg/min).

In this embodiment, the effective amount of the peptide or mimetic will preferably be in the range of 0.1 to 75 μg/kg/day, but more preferably in the range of about 0.1 to 25 μg/kg/day, still more preferably in the range of about 0.1 to 20 μg/kg/day or about 0.1 to 5 μg/kg/day, and even still more preferably, in the range of about 0.1 to 3.5 μg/kg/day or 0.1 to 1.5 μg/kg/day. Most preferably, the effective amount that is administered to the subject will be about 0.75 μg/kg/day.

The effective amount of the peptide or mimetic may, for example, be administered by infusion at a rate of ≦100 ng/kg/min, and preferably, at a rate of about ≦75 ng/kg/min, more preferably at a rate of about ≦50 ng/kg/min or ≦30 ng/kg/min, still more preferably at a rate of about ≦25 ng/kg/min, even still more preferably ≦20 ng/kg/min and, and yet still more preferably, at a rate of about ≦15 ng/kg/min. Most preferably, the effective amount is infused at a rate in the range of 1 to 15 ng/kg/min and, in one particularly preferred form of the method of the first embodiment, the effective amount of the peptide or mimetic is administered to the subject by infusion at a rate of about 12.5 ng/kg/min.

The effective amount of the peptide or mimetic may be infused (preferably, by intravenous (iv) or subcutaneous (sc) infusion) as a single dose per day or, otherwise, across multiple times per day (eg as three equal separate doses at 0 hours, 6 hours and 12 hours).

Typically, a single dose (or each dose of a multidose regimen), will be infused over a period of about 60 minutes.

Where the rate of the infusion is at a preferred rate of ≦75 ng/kg/min, it is considered that a single dose over a period of about 60 minutes will provide an effective daily dose (ie of ≦4.5 μg/kg/day). Further, where lower infusion rates are employed of 25 ng/kg/min and 12.5 ng/kg/min, it is considered that a single dose over a period of 60 minutes will provide an effective daily dose of ≦1.5 μg/kg/day and ≦0.75 μg/kg/day, respectively.

Thus, in a second embodiment, the present invention provides a method of treating chronic congestive heart failure (CHF) or acute decompensated congestive heart failure (ADCHF) in a subject, said method comprising administering to the subject an effective amount of a peptide derived from atrial natriuretic peptide (ANP) prohormone or a mimetic, wherein said effective amount is ≦4.5 μg/kg/day.

In this embodiment, the effective amount of the peptide or mimetic will preferably be in the range of 0.1 to 3.5 μg/kg/day, but more preferably in the range of about 0.1 to 1.5 μg/kg/day. Most preferably, the effective amount that is administered to the subject will be about 0.75 μg/kg/day.

Preferably, the method of the present invention is used for treating chronic CHF with a symptom severity level of NYHA class III or, more preferably, ADCHF with a symptom severity level of NYHA class III or IV. Most preferably, the method is used for treating ADCHF in a human characterised by:

1. Dyspnoea at rest or dyspnoea with minimal activity (ie difficulty breathing at rest while sitting, or difficulty breathing while lying flat or with one pillow, or difficulty breathing with minimal activity such as talking, eating); and
2. At least one of the following signs:
Tachypnoea with respiratory rate >20 breaths per minute, or
Pulmonary congestion/oedema with rales or crackles/crepitations at least a third above lung bases; and
   3. At least one of the following objective measures:
Pulmonary oedema/congestions as shown by chest X-ray, or
Circulating B-type natriuretic peptide (BNP) concentration of ≧400 pg/ml or circulating NT-proBNP concentration of ≧1000 pg/ml, or
A PCWP >20 mmHg or
A systolic dysfunction within the last 12 months (EF<50%) as determined by Trans-Thoracic Echocardiogram (TTE), nuclear testing, cardiac magnetic resonance imaging (MRI) or ventricular angiography).

The method utilises a peptide derived from ANP prohormone or a mimetic thereof.

It is to be understood that suitable peptides derived from ANP prohormone include the proANP 1-30, proANP 31-67 and proANP 79-98 peptides of human ANP prohormone described in U.S. Pat. No. 5,691,310, as well as derivatives of said peptides such as derivatives which include minor variations in the amino acid sequence which do not result in any substantial decrease or variation in biological activity. These variations may include conservative amino acid substitutions such as: Gly, Ala, Val, Ile, Leu, Met; Asp, Glu, Asn, Gln; Ser, Thr; Lys, Arg, His; Phe, Tyr, Trp, His; and Pro, Nα-alkylamino acids; and non-conservative amino acid substitutions. Some specific examples of suitable amino acid substitutions within the ANP prohormone sequence include: Ser→Arg (especially at position 39), Pro→Gln (especially at position 41), Thr→Ala (especially at position 59), Glu→Asp (especially at position 61), and Ser→Asn (especially at position 63).

Suitable mimetics of a peptide derived from ANP prohormone may be designed using any of the methods well known to the person skilled in the art for designing mimetics of peptides based upon amino acid sequences in the absence of secondary and tertiary structural information¹⁵. For example, peptide mimetic compounds may be produced by modifying amino acid side chains to increase the hydrophobicity of defined regions of the peptide (eg substituting hydrogens with methyl groups on aromatic residues of the peptides), substituting amino acid side chains with non-amino acid side chains (eg substituting aromatic residues of the peptides with other aryl groups), and substituting amino- and/or carboxy-termini with various substituents (eg substituting aliphatic groups to increase hydrophobicity). Alternatively, mimetic compounds of a peptide derived from ANP prohormone may be a so-called peptoid (ie non-peptide) which includes modification of the peptide backbone (ie introducing amide bond surrogates by, for example, replacing the nitrogen atoms in the backbone with carbon atoms), or includes N-substituted glycine residues, one or more D-amino acids (in place of L-amino acid(s)) and/or one or more α-amino acids (in place of β-amino acids or γ-amino acids). Further, suitable mimetic compounds of a peptide derived from ANP prohormone include “retro-inverso peptides” where the peptide bonds are reversed and D-amino acids assembled in reverse order to the order of the L-amino acids in the peptide sequence upon which the mimetic is based, and other non-peptide frameworks such as steroids, saccharides, benzazepine 1,3,4-trisubstituted pyrrolidinone, pyridones and pyridopyrazines.

Preferably, the method of the present invention comprises the administration of proANP 31-67 (ie a peptide consisting of the amino acids 31-67 of the ANP prohormone), known as vessel dilator (VSDL), or a derivative thereof. The amino acid sequence of the preferred, human, VSDL is as follows:

(SEQ ID NO: 1)
Glu-Val-Val-Pro-Pro-Gln-Val-Leu-Ser-Glu-Pro-Asn-
Glu-Glu-Ala-Gly-Ala-Ala-Leu-Ser-Pro-Leu-Pro-Glu-
Val-Pro-Pro-Trp-Thr-Gly-Glu-Val-Ser-Pro-Ala-Gln-
Arg.

Other suitable VSDL peptides include:

Pongo pygmaeus (common orang-utan)
(SEQ ID NO: 2)
Glu-Val-Val-Pro-Pro-Gln-Val-Leu-Ser-Glu-Gln-Asn-
Glu-Glu-Ala-Gly-Ala-Ala-Leu-Ser-Pro-Leu-Pro-Glu-
Val-Pro-Pro-Trp-Thr-Gly-Glu-Val-Ser-Pro-Ala-Gln-
Arg;
Macaca mulatta (rhesus monkey)
(SEQ ID NO: 3)
Glu-Val-Val-Pro-Pro-Gln-Val-Leu-Arg-Glu-Gln-Asn-
Glu-Glu-Ala-Gly-Ala-Ala-Leu-Ser-Pro-Leu-Pro-Glu-
Val-Pro-Pro-Trp-Thr-Gly-Asp-Val-Ser-Pro-Ala-Gln-
Arg;
and
Felis catus
(SEQ ID NO: 4)
Glu-Val-Val-Pro-Pro-Gln-Val-Leu-Ser-Glu-Gln-Asn-
Glu-Glu-Ala-Gly-Ala-Ala-Leu-Ser-Pro-Leu-Pro-Glu-
Val-Pro-Pro-Trp-Ala-Gly-Glu-Val-Asn-Pro-Ala-Gln-
Arg.

Peptides derived from ANP prohormone may be produced by any of the standard protein synthesis methods well known to the person skilled in the art or, more preferably, by recombinant techniques involving, for example, the introduction of a polynucleotide molecule encoding the particular peptide into a suitable host cell (eg a host cell selected from bacterial cells such as E. coli, Streptomyces and S. typhimurium; fungal cells such as yeast cells; insect cells such as Drosophila S2 and Spodoptera Sf9 cells; animal cells such as Chinese hamster ovary (CHO), monkey kidney (COS) cells and human embryonic kidney 293 (HEK 293) cells; and plant cells) and culturing the cell under conditions suitable for the expression of the particular peptide.

Preferably, the peptide derived from ANP prohormone or a mimetic thereof will be administered as a composition consisting of a simple solution or suspension of the peptide or mimetic in a pharmaceutically-acceptable carrier. However, it will be readily appreciated by the person skilled in the art, that the peptide or mimetic may be bound or associated with a carrier molecule (eg a carrier protein or fusion partner such as human serum albumin (HSA) or a polysaccharide (eg Dextran) or polyether (eg polyethylene glycol)) in order to modulate the biological activity and/or serum half-life time of the peptide or mimetic.

The term “pharmaceutically-acceptable carrier” as used herein refers to any pharmaceutically- or veterinary-acceptable solvent, suspending agent or vehicle for delivering the peptide derived from ANP prohormone or mimetic thereof to the subject. The carrier may include one or more pharmaceutical additives of a type appropriate to, for example, iv administration (eg excipients, preservatives, stabilisers etc).

The peptide derived from ANP prohormone or a mimetic thereof may be administered to the subject in a combination therapy. However, while it is considered that the administration of such a peptide (eg VSDL) or mimetic thereof should not cause clinically significant hypotension, they can be vasodilatory and, therefore, it is preferred that any combination therapy avoids other vasodilatory agents which may cause a synergetic blood pressure lowering. For that reason, the method of the present invention may not be suitable for subjects already using vasodilatory agents.

The administration of the peptide derived from ANP prohormone or a mimetic thereof by infusion is, preferably, achieved intravenously (iv) (which is particularly suitable in the hospital setting) or subcutaneously (sc) (which is suitable for both hospitalised patient and out-of-hospital administration). For example, infusion may be via a standard catheter or implantable drug port (eg a Port-a-Cath®; Smiths Medical MD, Inc., St. Paul Minn., United States of America), or otherwise achieved using a drug infusion pump (eg implantable drug infusion pumps such as an Alzet® osmotic pump (Durect Corporation, Cupertino Calif., United States of America) and a Duros® device (Intarcia Therapeutics, Inc., Hayward Calif., United States of America), or a drug infusion pump for subcutaneous (sc) administration such as a Paradigm™ device (Medtronic Australasia Pty Ltd, Gladesville NSW, Australia) all of which can provide a controlled release of the peptide or mimetic) which preferably infuses the peptide or mimetic at a constant rate.

The use of an implantable drug port or drug infusion pump will be particularly well suited for a long term treatment method according to the present invention. Typically, the peptide or mimetic will be infused at a constant rate, however, in some cases it may be desirable to employ a drug infusion pump employing a feedback control mechanism (eg a feedback linked to measurement of oedema (in the lung) or other surrogate marker) to control release of the peptide or mimetic.

In one particularly preferred embodiment, the invention provides a method of treating ADCHF in a subject, said method comprising administering to the subject an effective amount of VSDL, wherein said effective amount is in the range of about 0.1 to 20 μg/kg/day and is administered by infusion, preferably intravenous (iv) or subcutaneous (sc) infusion, at a rate of ≦50 ng/kg/min.

In another particularly preferred embodiment, the invention provides a method of treating ADCHF in a subject, said method comprising administering to the subject an effective amount of VSDL, wherein said effective amount is in the range of about 0.1 to 1.5 μg/kg/day and is administered by infusion, preferably intravenous (iv) or subcutaneous (sc) infusion.

It will be appreciated by the person skilled in the art that numerous variations and/or routine modifications may be made to the method of the present invention without departing from the spirit or scope of the invention as broadly described. For example, it will be understood that the effective amount and frequency of administration of the peptide or mimetic for any particular subject may be varied and will depend upon a variety of factors including the activity of the particular peptide or mimetic that is utilised, the metabolic stability and length of action of the particular peptide or mimetic, the age, body weight, general health, sex and diet of the particular subject, and the time of administration, rate of excretion, drug combination and severity of the chronic CHF or ADCHF being treated.

The present invention further provides the use of a peptide derived from atrial natriuretic peptide (ANP) prohormone or a mimetic thereof in the manufacture of a medicament for infusion at a rate of about 1 to 400 ng per kg subject body weight per minute (ng/kg/min), for treating chronic congestive heart failure (CHF) or acute decompensated congestive heart failure (ADCHF) in a subject. In such use, preferably, the medicament is capable of providing an effective amount of the peptide or mimetic in the range of about in the range of 0.1 to 75 μg/kg/day, but more preferably in the range of about 0.1 to 25 μg/kg/day, still more preferably in the range of about 0.1 to 20 μg/kg/day or about 0.1 to 5 μg/kg/day, and even still more preferably, in the range of about 0.1 to 3.5 μg/kg/day or 0.1 to 1.5 μg/kg/day.

Alternatively, the present invention provides the use of a peptide derived from atrial natriuretic peptide (ANP) prohormone or a mimetic thereof in the manufacture of a medicament for treating chronic congestive heart failure (CHF) or acute decompensated congestive heart failure (ADCHF) in a subject, wherein said medicament is adapted to provide said subject with an effective amount of said peptide or mimetic of ≦4.5 μg/kg/day. In such use, preferably, the medicament is capable of providing an effective amount of the peptide derived from atrial natriuretic peptide (ANP) prohormone or a mimetic in the range of 0.1 to 3.5 μg/kg/day, but more preferably in the range of about 0.1 to 1.5 μg/kg/day.

Most preferably, in the use of the present invention, the medicament is capable of providing an effective amount of the peptide or mimetic of about 0.75 μg/kg/day.

The medicament may, for example, be adapted for infusion at a rate of <100 ng/kg/min, and preferably at a rate of about ≦75 ng/kg/min, more preferably at a rate of about ≦50 ng/kg/min or ≦30 ng/kg/min, still more preferably at a rate of about ≦25 ng/kg/min, even still more preferably ≦20 ng/kg/min and, and yet still more preferably at a rate of about ≦15 ng/kg/min. Most preferably, the medicament is adapted for infusion at a rate of about 12.5 ng/kg/min.

The medicament may be used in a single dose per day or administered across multiple times per day (eg 2 to 4 times per day). Typically, a single dose (or each dose of a multidose regimen) will be administered over a period of about 60 minutes.

Preferably, the medicament is intended for treating the particular indication of ADCHF with a symptom severity level of NYHA class III or IV.

Preferably, the use of the present invention is a use of VSDL or a derivative thereof.

The medicament may be infused intravenously (iv) via a standard catheter, implantable drug port or drug infusion pump, or subcutaneously (sc) via a drug infusion pump.

In one particularly preferred embodiment, the invention provides the use of VSDL in the manufacture of a medicament for infusion at a rate of ≦50 ng/kg/min, for treating ADCHF in a subject.

In another particularly preferred embodiment, the invention provides the use of VSDL in the manufacture of a medicament for treating ADCHF in a subject, wherein said medicament is adapted to provide said subject with an effective amount of said peptide or mimetic in the range of about 0.1 to 1.5 μg/kg/day.

The present invention still further provides an infusion device containing, or provided with, a medicament comprising a peptide derived from atrial natriuretic peptide (ANP) prohormone or a mimetic thereof adapted for infusion at a rate of about 1 to 400 ng per kg subject body weight per minute (ng/kg/min), for treating chronic congestive heart failure (CHF) or acute decompensated congestive heart failure (ADCHF) in a subject.

Alternatively, the present invention provides an infusion device containing, or provided with, a medicament comprising a peptide derived from atrial natriuretic peptide (ANP) prohormone or a mimetic thereof for treating chronic congestive heart failure (CHF) or acute decompensated congestive heart failure (ADCHF) in a subject, wherein said medicament is adapted to provide said subject with an effective amount of said peptide or mimetic of ≦4.5 μg/kg/day.

The infusion device may be selected from devices for intravenous (iv) infusion (eg a standard catheter, implantable drug port or iv drug infusion pump) or subcutaneous (sc) infusion (eg sc drug infusion pump).

The medicament, which may be as described above, may be contained within a reservoir provided within the device or which may otherwise be operably connected thereto. The arrangement of a connectable reservoir may be such that it is only capable of being reasonably used with the said device.

In one particularly preferred embodiment, the invention provides an infusion device containing, or provided with, a medicament comprising VSDL adapted for infusion at a rate of ≦50 ng/kg/min, for treating ADCHF in a subject.

In another particularly preferred embodiment, the invention provides an infusion device containing, or provided with, a medicament comprising VSDL adapted to provide said subject with an effective amount of said peptide or mimetic in the range of about 0.1 to 1.5 μg/kg/day, for treating ADCHF in a subject.

In a further aspect, the present invention provides the use of a peptide derived from atrial natriuretic peptide (ANP) prohormone or a mimetic thereof in the treatment of chronic congestive heart failure (CHF) or acute decompensated congestive heart failure (ADCHF) in a subject, characterised in that the peptide is infused into the subject at a rate of about 1 to 400 ng per kg subject body weight per minute (ng/kg/min).

In a still further aspect, the present invention provides the use of a peptide derived from atrial natriuretic peptide (ANP) prohormone or a mimetic thereof in the treatment of chronic congestive heart failure (CHF) or acute decompensated congestive heart failure (ADCHF) in a subject, characterised in that the peptide is administered into the subject at an effective amount of ≦4.5 μg/kg/day.

The present invention is hereinafter further described by way of the following, non-limiting example(s).

### EXAMPLE(S)

### Example 1

### Treatment of ADCHF with iv Administration of Vessel Dilator

This example describes a study to examine whether VSDL administration to ADCHF patients will be safe and induce improvements in haemodynamic parameters, as well as renal, natriuretic and diuretic parameters, whilst regulating plasma volume and BP within clinically acceptable ranges and without seriously adverse side effects.

### Methods and Materials

Formulation VSDL in the form of a white lyophilised powder (synthesised using standard protein synthesis method by Auspep Pty Ltd, Parkville, VIC, Australia), stored in an ultra low freezer (−80° C.), was reconstituted in a vial with 10 ml of 0.9% saline (preservative free) and aseptically transferred into a 20 ml syringe (that connects to a patient cannula) before use.

### Dosage

Initially, an iv dose of 12.5 ng/kg/min was infused into test subjects for 60 minutes with a syringe driver (Alaris Asena GS syringe driver; CareFusion Corporation, San Diego, Calif., United States of America). This “safety dose” was chosen merely to trial the subjects for any adverse effects. Subsequently, a dose of 25 ng/kg/min (as infused for 60 minutes) was trialled. Additionally, a trial will be conducted with a dose of 50 ng/kg/min (for 60 minutes).

### Study Population

Test adult subjects, both male and female, showing either acute exacerbations of chronic CHF or ADCHF (ie in individuals who had not previously shown heart failure), were recruited for the study. The following inclusion and exclusion criteria were applied:

### Inclusion Criteria

In order to be eligible, a subject must have chronic CHF or ADCHF (NYHA class III-NYHA class IV) defined as:

1. Showing dyspnoea at rest or dyspnoea with minimal activity (ie difficulty breathing at rest while sitting, or difficulty breathing while lying flat or with one pillow, or difficulty breathing with minimal activity such as talking, eating); AND
2. Showing at least one of the following signs:
Tachypnoea with respiratory rate >20 breaths per minute, OR
Pulmonary congestion/oedema with rales or crackles/crepitations at least a third above lung bases; AND
   3. At least one of the following objective measures:
Showing, by chest X-ray, pulmonary oedema/congestions OR
Circulating B-type natriuretic peptide (BNP) concentration of ≧400 pg/ml or circulating NT-proBNP concentration of ≧1000 pg/ml at presentation, OR
PCWP >20 mmHg OR
Showing systolic dysfunction within the last 12 months (EF<50%) as determined by TTE, nuclear testing, cardiac MRI or ventricular angiography);
   and be:
   4. Male and/or female, 18 years or older; and
   5. If a woman of child bearing potential, testing negative to β-hCG.

### Exclusion Criteria:

1. Evidence in the ED for Myocardial Infarction (MI) or high risk acute coronary syndrome within past 6 weeks, as determined by creatinine kinase (CK)/creatinine kinase muscle-brain isoenzyme (CK-MB) ≧2 times upper limit of normal or elevation of Troponin T at baseline >0.1 or as determined by Trans-Thoracic electrocardiogram (TTE);
2. Hypotension (SBP<90 mmHg), cardiogenic shock, volume depletion;
3. Persistent, uncontrolled hypertension (SBP >180 mm Hg);
4. Subjects with the presence of any CMR contra-indication (including PPM, cerebral aneurysm clips or non-MRI approved metallic implant) are excluded from any MRI study component only;
5. Congenital heart defects (including Ventricular Septal Defect, Atrial Septal Defect, Patent Ductus Arteriosus, Tetralogy of Fallot, and Tricuspid Atresia);
6. Cardiac surgery within past 4 weeks;
7. Diastolic heart failure (preserved left ventricular function—determined by known history, ie E:E prime ratio >15);
8. Severe valvular heart disease: Aortic Stenosis <1.0 cm², any Idiopathic hypertrophic subaortic stenosis or Hypertrophic Obstructive Cardiomyopathy, acute Aortic Regurgitation grade 3 or 4 and Mitral Regurgitation grade 3 or 4;
9. History of cerebrovascular accident (within past 4 weeks) as determined by MRI or Computerised Tomography (CT) Scan;
10. Acute or chronic active infection, including pneumonia and urinary tract infection documented by appropriate culture result;
11. Very significant renal impairment as determined by a creatinine clearance of <20 ml/min; and
12. Prior participation in any other clinical trial within past 30 days, including present day.

Three subjects were provided with a single dose of VSDL of 12.5 ng/kg/min for 60 minutes (ie single dose regimen) for a daily dose of 0.75 μg/kg. Subsequently, a further subject was trialled with a single dose of VSDL of 25 ng/kg/min for 60 minutes (to achieve a daily dosage of 1.5 μg/kg). Two further subjects are/have been recruited for trials with a single dose of VSDL of 25 ng/kg/min for 60 minutes, along with another subject (in a preliminary trial) for dosing with a single infusion of VSDL of 50 ng/kg/min for 60 minutes (to achieve a dosage of 3 μg/kg). The results will be compared with subjects recruited as a control group and who will receive a standard method of care for the treatment of ADCHF.

### Assessment of Efficacy

Treated subjects were assessed by independent reviewers.

Markers of efficacy were measured at baseline (prior to VSDL infusion), 1 hour (post VSDL infusion), and 24 hours. The markers included CI, PCWP, pulmonary arterial wedge pressure (PAWP), BP, SVR and PVR, and were measured using standard methods well known to the person skilled in the art. Troponin T, proBNP, CK and CK-MB concentrations were also measured by standard methods.

Secondary efficacy variables that were measured included: dyspnea relief, and increased urine flow and volume output.

### Results

Subjects given 12.5 ng VSDL per kg/min for 60 mins

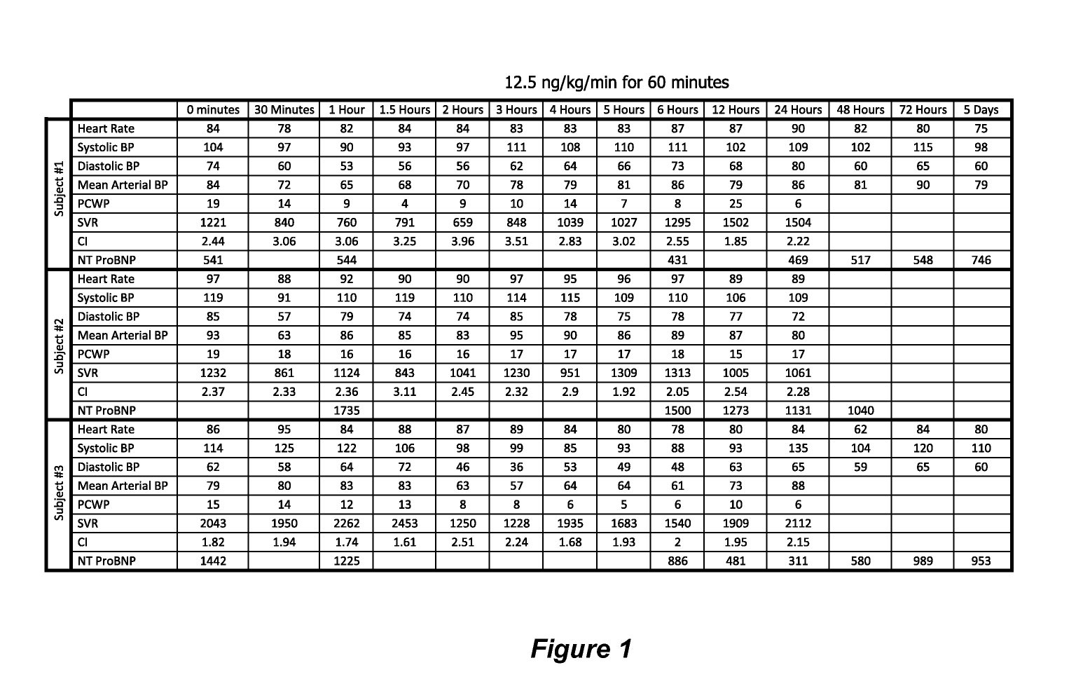

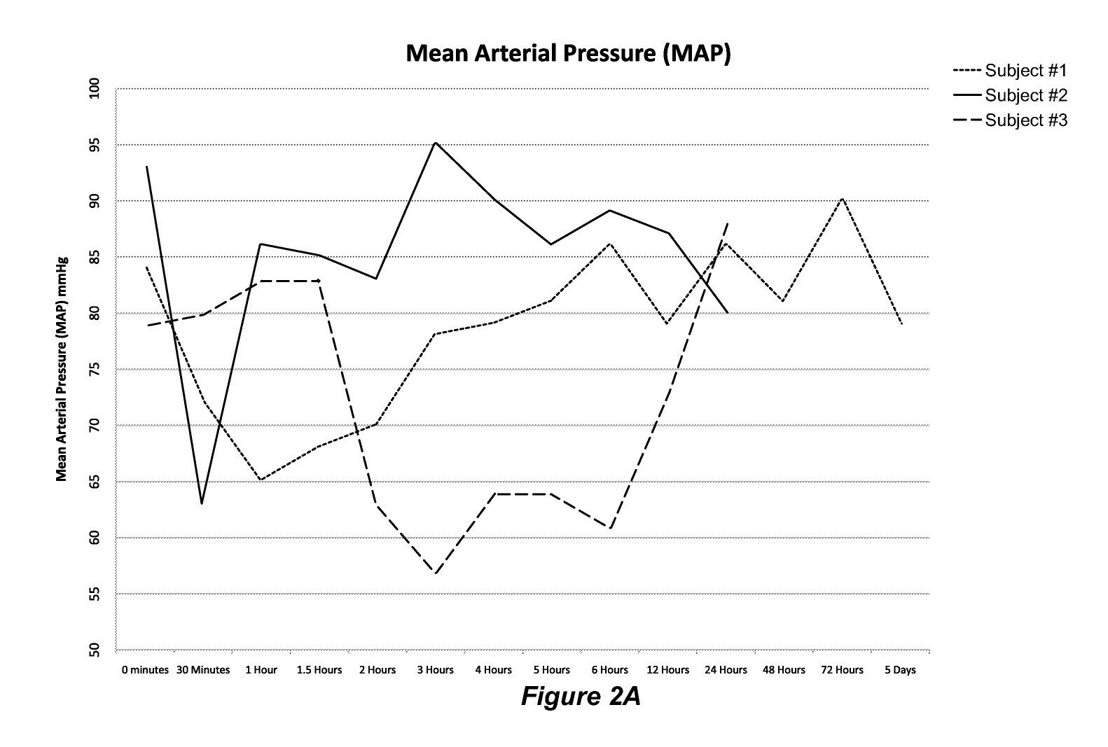

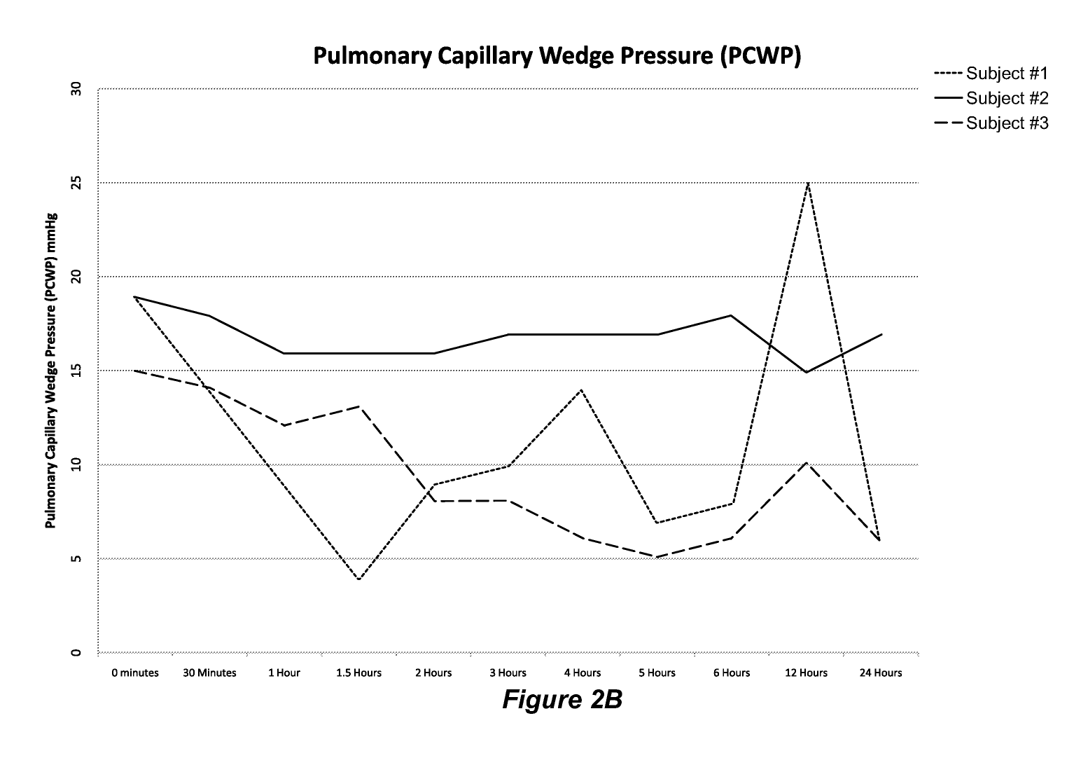

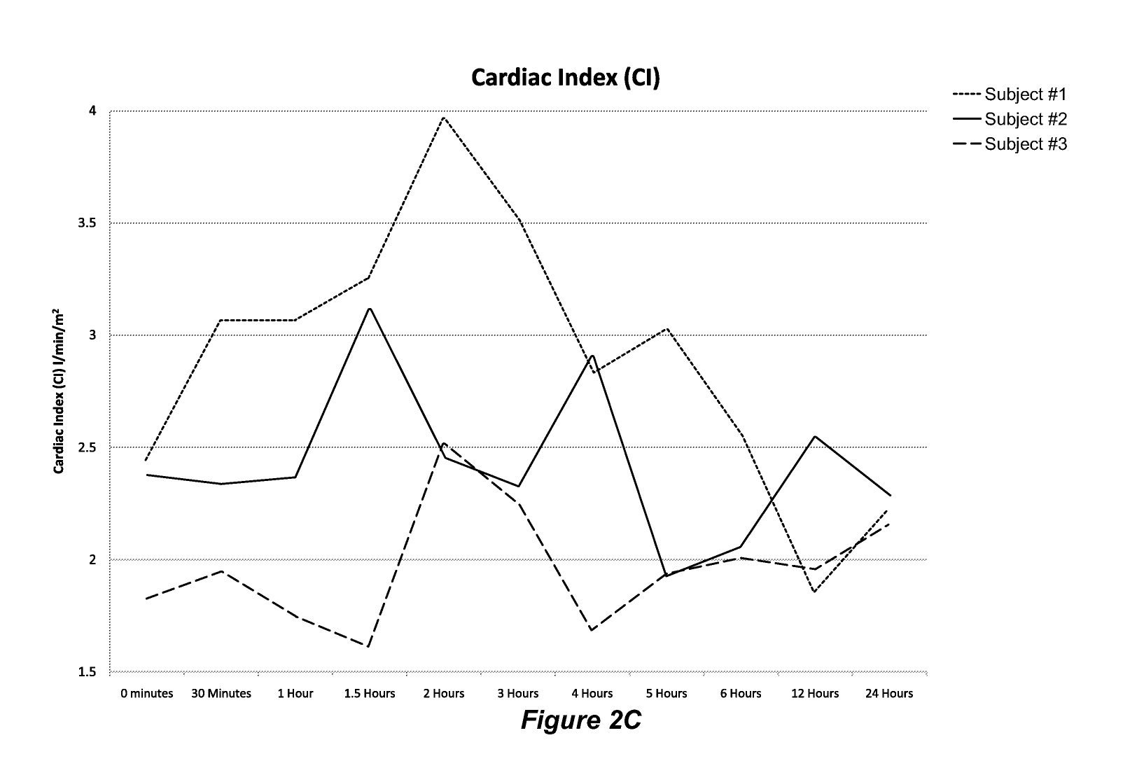

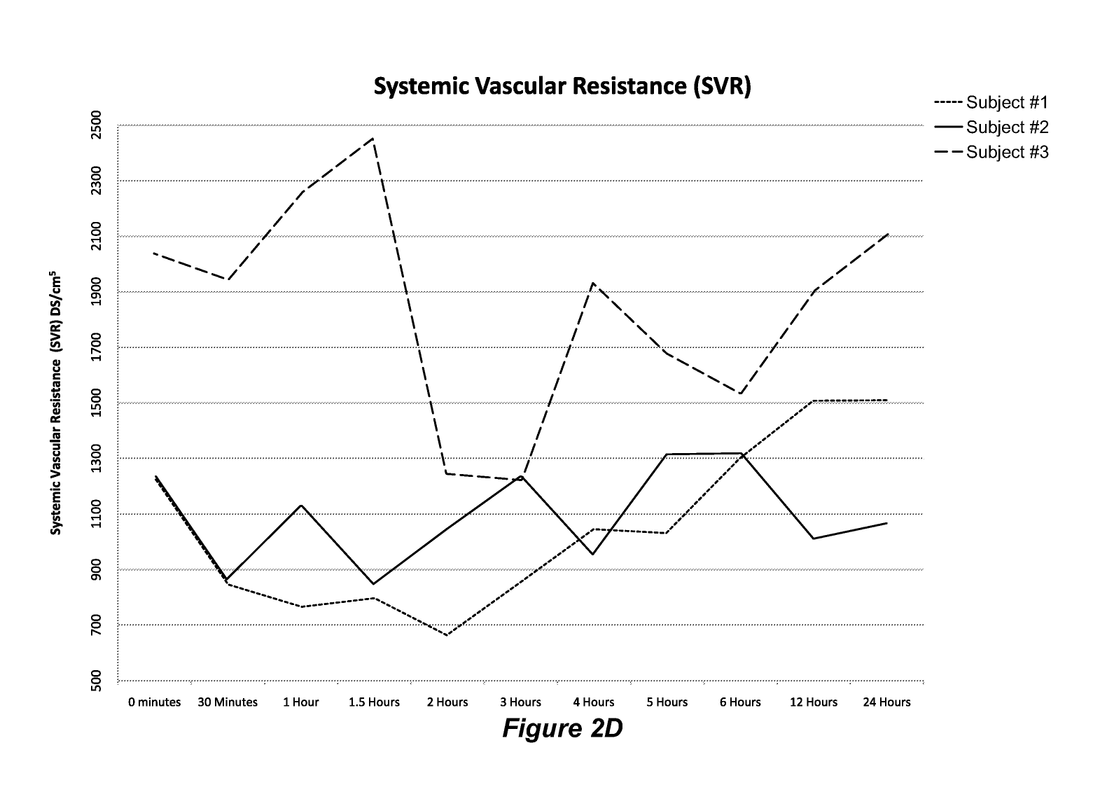

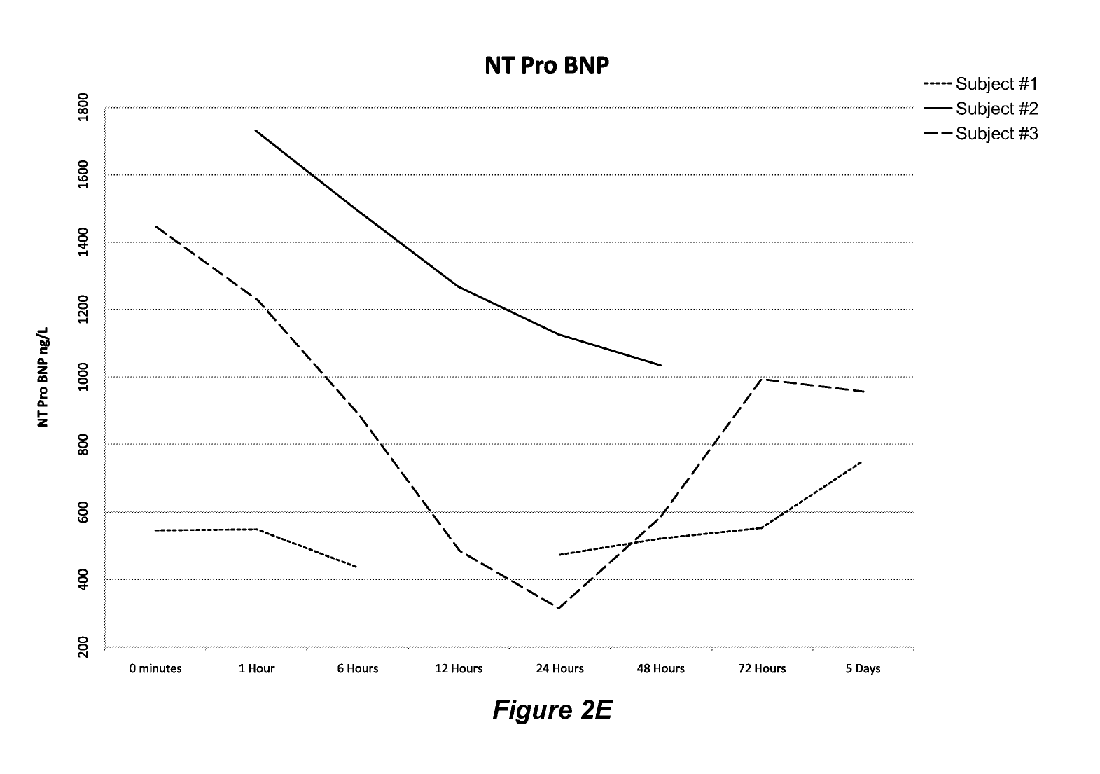

The results shown by subjects treated with a single VSDL dose of 12.5 ng/kg/min for 60 mins are provided in FIGS. 1 and 2 and summarised in Table 1. The reviewers confirmed that they saw no safety risks.

Subjects given 25 ng VSDL per kg/min for 60 mins

The results shown by a subject treated with a single VSDL dose of 25 ng/kg/min for 60 mins are provided in Table 2. There were marked beneficial effects; in particular, decreasing dyspnea (ie from 3+ to 1+), increased CI, decreased systemic vascular resistance (SVR) and peripheral vascular resistance (PVR), and decreased PAWP. The infusion was well tolerated and no side effects were observed. The reviewers confirmed that they saw no safety risks.

TABLE 1

| | Subject #1 | Subject #2 | Subject #3 |
|---|---|---|---|
| Summary of | This male subject | This male subject | This female subject |
| observations | received a single dose | received a single dose | received a single dose |
| | of iv VSDL in saline | of iv VSDL in saline | of iv VSDL in saline |
| | (12.5 ng/kg/min for 60 | (12.5 ng/kg/min for 60 | (12.5 ng/kg/min for 60 |
| | mins), on 28 Aug. | mins), on 1 Sep. | mins), on 7 Oct. |
| | 2010. The subject | 2010. The infusion was | 2010. The subject |
| | tolerated the infusion | well tolerated by the | tolerated the infusion |
| | and was | subject. There was | well. There was an |
| | uncompromised during | marked diuresis in the | increase in CI, a fall in |
| | and post infusion. The | first 30 mins following | PCWP, and an |
| | subject responded | infusion and dyspnea | asymptomatic drop in |
| | during the infusion with | disappeared within 1 | BP. It also appeared |
| | an increase in cardiac | hour of starting | that SVR was |
| | index (CI), a drop in | infusion. There was no | improved. Further, the |
| | pulmonary capillary | significant change in | blood concentration of |
| | wedge pressure | CI, PCWP or mean BP. | the CHF marker |
| | (PCWP), and an | However, SVR | proBNP, decreased |
| | asymptomatic drop in | decreased 25% in the | 75% during and within |
| | blood pressure (BP). | first 30 mins of the | the 24 hours following |
| | | infusion; the VSDL | the infusion. The |
| | | therefore caused | subject also remained |
| | | beneficial effects in the | clinically stable during |
| | | treatment of acute heart | this period. |
| | | failure in this subject | |
| | | with global dyskinesis | |
| | | and no side-effects. The | |
| | | subject remained stable | |
| | | during the 24 hours | |
| | | following the infusion. | |

TABLE 2

| | |
|---|---|
| Baseline | 24 hrs post-infusion |

| | | | | |
|---|---|---|---|---|
| CI | 2.26 | L/min/m² | 2.34 | L/min/m² |
| PAWP | 22 | mmHg | 6 | mmHg |
| Arterial BP | 124 | mmHg | 96 | mmHg |
| (mean) | | | | |
| Urinary | | | 2170 | ml |
| Output | | | | |
| Troponin T | <0.02 | μg/L | <0.02 | μg/L |
| CK | 188 | U/L | 156 | U/L |
| CK-MB | 2.0 | μg/L | 1.3 | μg/L |

### Discussion

Since it was thought that subjects suffering significant CHF are unable to produce adequate endogenous amounts of natriuretic peptides to compensate for congestion, the present applicant had selected doses of exogenous VSDL infusion of 50 ng/kg/min, 100 ng/kg/min and 200 ng/kg/min for 60 minutes to ascertain the approximate amounts of exogenous VSDL required by late NHYA class III-NYHA class IV patients to reinstate homeostasis. Such doses were expected to result in an approximately 2-fold, 4-fold and 8-fold increase in total circulating VSDL, respectively. Further, the present applicant had particularly proposed an exogenous dose of VSDL at <100 ng/kg/min (for 60 mins) for the treatment of chronic CHF or ADCHF as a safe and effective dosage, since it has been identified that the release rate from the heart of VSDL with physiological stimuli is about 138 ng/kg body weight per minute¹⁶ and similar dosages have been previously observed to cause a marked natriuresis and diuresis in healthy humans^{17, 18} and, also, improved haemodynamic and renal parameters in persons with stable NYHA class III chronic CHF¹⁰. The molar equivalent of the 100 ng/kg body weight dose (for 60 mins) is 26 pmol/kg body weight (for 60 mins). However, in the preliminary trials described above, it was surprisingly found that beneficial effects for the treatment of chronic CHF can be achieved at doses that are much less than 100 ng/kg/min for 60 mins. In particular, at 12.5 ng/kg/min (for 60 mins) and 25 ng/kg/min (for 60 mins) One reviewer commented in respect of Subject #3 given 12.5 ng/kg/min (for 60 mins), that the observed 75% drop in the concentration of proBNP (a CHF marker) within 24 hours of the infusion was “truly spectacular”.

### Example 2

### Estimation of Baseline and Cₘₐₓ for VSDL Following Infusion

Following the surprising finding that beneficial effects for the treatment of chronic CHF can be achieved with doses that are much less than that which had been proposed, a study was undertaken to estimate the baseline levels of VSDL and Cₘₐₓ (ie the peak VSDL concentration that is achieved) following infusion.

### Methods and Materials

Cₘₐₓ was estimated from blood samples of a CHF patient (NYHA class III) taken at 30 mins, 1 hr, 1.5 hr, 2 hr, 3 hr, 4 hr, 5 hr, 6 hr and 12 hr following infusion, using a proprietary method (CPR Pharma Services Pty Ltd, Thebarton, SA, Australia) involving mass spectroscopy. The baseline VSDL concentration (ie endogenous level) was estimated from an identical sample taken prior to infusion (ie “predose”).

### Results

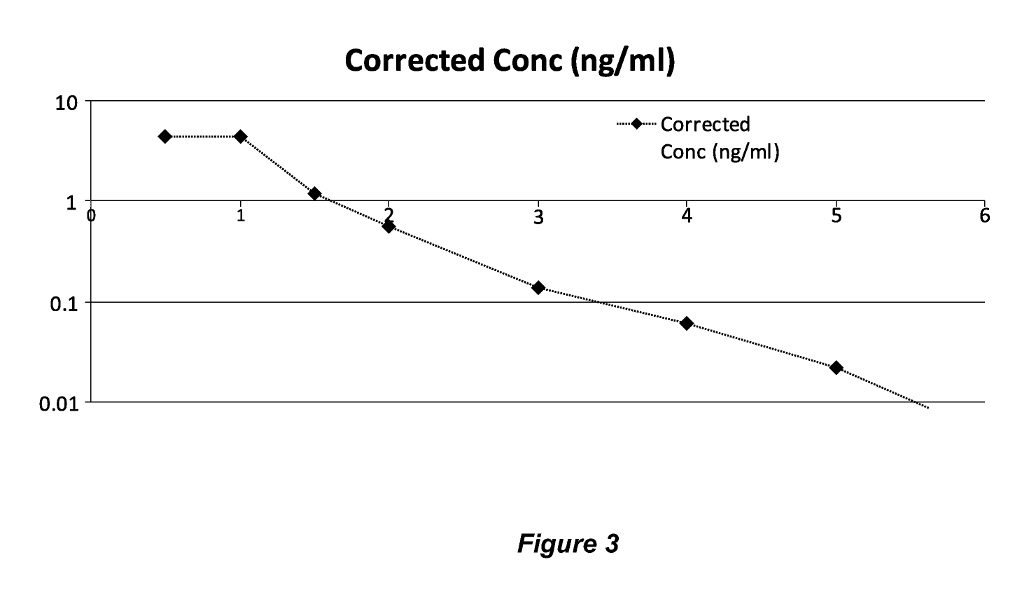

The baseline VSDL concentration was estimated as 0.024 ng/ml. The measured VSDL concentrations in the samples taken at the various time points are shown in Table 3 and FIG. 3.

A corrected concentration, which accounts for the baseline concentration is also shown; however due to the low level of the baseline concentration, the corrected concentrations vary little from the actual, measured concentrations.

The Cₘₐₓ was therefore 4.376 ng/ml (˜4.4 ng/ml) achieved 30 mins (ie Tₘₐₓ of 0.5 hr) after infusion. This value was also estimated after 1 hr. There is a slight curve to the measured VSDL concentration profile (FIG. 3) indicating that the elimination of VSDL from the blood stream does not clearly follow first order kinetics. Using the values shown in Table 3 to estimate the terminal elimination rate, the LOQ of 0.2 ng/ml gives an estimated half-life (t_{1/2}) of 20 minutes, while using the point just below the LOQ estimates a slightly longer half-life of 25-30 minutes, excluding and including the Cₘₐₓ values, respectively.

TABLE 3

| | Corrected | |
|---|---|---|
| | Concentration | Concentration |
| Time point (hrs) | (ng/ml) | (ng/ml) |
| 0.5 | 4.376 | 4.4 |
| 1 | 4.376 | 4.4 |
| 1.5 | 1.176 | 1.2 |
| 2 | 0.556 | 0.58 |
| 3 | 0.136 | 0.16 |
| 4 | 0.061 | 0.085 |
| 5 | 0.022 | 0.046 |
| 6 | 0.005 | 0.029 |
| 12 | 0.018 | 0.042 |
| Predose | 0.024 | 0.024 |

### Discussion

The baseline VSDL concentration in blood was estimated as 0.024 ng/ml. This was surprisingly low given previously published reports of endogenous basal levels of circulating VSDL in patients with varying degrees of CHF; for example, a basal VSDL concentration of 3412±546 (SEM) fmol/ml has been reported for CHF NYHA class III patients and 7691±1344 (SEM) fmol/ml for CHF NYHA class IV patients⁶. These values correspond to about 13.23 ng/ml and 29.82 ng/ml. Cₘₐₓ for the infused VSDL was achieved at a Tₘₐₓ of 0.5 hr. Further, the t_{1/2} of 25-30 mins estimated from this study is much shorter than that previously reported (ie 120 mins). While not wishing to be bound by theory, it is considered that this previously reported t_{1/2} of 120 mins may have been the result of inadvertently providing a supramaximal dose.

The surprising finding that effective treatment of chronic CHF or ADCHF patients with VSDL can be achieved with doses that are substantially lower than expected, is likely to have important implications in Chronic CHF and ADCHF treatment including, for example, avoidance of seriously adverse side effects as well as allowing treatment of patients with significant renal impairment (eg having a creatinine clearance of 20 to 60 ml/min) whom might otherwise may not have been regarded as treatable.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

All publications mentioned in this specification are herein incorporated by reference. Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed in Australia or elsewhere before the priority date of each claim of this application.

It will be appreciated by the person skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

### REFERENCES

1. Heart Disease and Stroke Statistics-2003 update. Dallas: American Heart Association 2002.
2. Heywood J T, Heart Fail Rev 9:195-201 (2004).
3. Fonarow G, Heart Fail Rev 9:179-185 (2004).
4. Vesely D L, Am J Physiology 285:F167-F177 (2003).
5. Habibullah A A et al., Clin Exp Pharmacol Physiol 22:130-135 (1995).
6. Winters C J et al., Circulation 80:438-449 (1989).
7. Vesely D L et al., Proc Soc Exp Biol Med 192:230-235 (1989).
8. Hunter E F M et al., Scan J Clin Lab Invest 58:205-216 (1998).
9. Franz M et al., Kidney Int 58:374-378 (2000).
10. Vesely D L et al., Circulation 98:323-329 (1998).
11. Vesely D L et al., Life Sci 66(10):905-913 (2000).
12. De Palo E F et al., Clin Chem 46:843-847 (2000).
13. Franz M et al., Kidney Int 59:1928-1934 (2001).
14. Peacock W F and M D Emerman, Heart Failure Reviews 9:187-193 (2004).
15. Kirshenbaum K et al., Curr Opin Struct Biol 9:530-535 (1999).
16. Vesely D L et al., J Clin Endocrinol Metab 78:1128-1134 (1994).
17. Vesely D L et al., Circulation 90:1129-1140 (1994).
18. Vesely D L et al., Peptides 11:193-197 (1990).
19. Fonarow G C et al., Arch Intern Med 168(8):847-854 (2008).

1. A method of treating chronic congestive heart failure (CHF) or acute decompensated congestive heart failure (ADCHF) in a subject, said method comprising administering to the subject an effective amount of vessel dilator (VSDL) or a mimetic thereof in the range of about 0.1 to 5 μg/kg/day.

2. The method of claim 1, wherein the effective amount of the VSDL or mimetic is in the range of about 0.1 to 3.5 μg/kg/day.

3. The method of claim 1, wherein the VSDL or mimetic is administered by infusion at a rate of ≦50 ng/kg/min.

4. The method of claim 1, wherein the effective amount of the VSDL or mimetic is in the range of 0.1 to 1.5 μg/kg/day.

5. The method of claim 4, wherein the VSDL or mimetic is administered by infusion at a rate of about 25 ng/kg/min.

6. The method of claim 1, wherein the effective amount of the VSDL or mimetic is administered as a single daily dose by infusion over a period of about 60 minutes.

7. The method of claim 1, wherein the method is used for treating chronic CHF with a symptom severity level of NYHA class III.

8. The method of claim 1, wherein the method comprises administering VSDL.

9. The method of claim 8, wherein the VSDL consists of the amino acid sequence shown as SEQ ID NO: 1.

10. A method of treating acute decompensated congestive heart failure (ADCHF) in a subject, said method comprising administering to the subject an effective amount of vessel dilator (VSDL) or mimetic thereof.

11. The method of claim 10, wherein the VSDL or mimetic is administered by infusion at a rate of 1 to 400 ng per kg subject body weight per minute (ng/kg/min).

12. The method of claim 11, wherein the effective amount of the VSDL or mimetic is in the range of 0.1 to 75 μg/kg/day.

13. The method of claim 10, wherein said effective amount is in the range of about 0.1 to 20 μg/kg/day and is administered by infusion at a rate of ≦50 ng/kg/min.

14. The method of claim 10, wherein said effective amount is in the range of about 0.1 to 1.5 μg/kg/day and is administered by infusion.

15. The method of claim 10, wherein the method comprises administering VSDL.

16. The method of claim 15, wherein the VSDL consists of the amino acid sequence shown as SEQ ID NO: 1.

17. An infusion device containing, or provided with, a medicament comprising vessel dilator (VSDL) or a mimetic thereof adapted for infusion of the VSDL or mimetic, for treating chronic congestive heart failure (CHF) or acute decompensated congestive heart failure (ADCHF) in a subject, wherein said device is adapted to administer a dose of the VSDL or mimetic in the range of about 0.1 to 5 μg/kg/day.

18. The device of claim 17, adapted to administer a dose of the VSDL or mimetic in the range of about 0.1 to 3.5 μg/kg/day.

19. The device of claim 17, adapted to infuse the VSDL or mimetic at a rate of ≦50 ng/kg/min.

20. The device of claim 17, adapted to administer a dose of the VSDL or mimetic in the range of 0.1 to 1.5 μg/kg/day.

21. The device of claim 17, adapted to infuse the VSDL or mimetic as a single daily dose over a period of about 60 minutes.

22. The device of claim 17, wherein the VSDL or mimetic is contained within a reservoir provided within the device or which is otherwise operably connected thereto.

23. The device of claim 22, wherein VSDL is contained within the reservoir.

24. The device of claim 23, wherein the VSDL consists of the amino acid sequence shown as SEQ ID NO: 1.
